(19) United States
(12) Patent Application Publication
(10) Pub. No.: US 2026/0305982 A1
(43) Pub. Date: Oct. 8, 2026

(54) REACTION FORCE MEASURING PLATE AND REACTION FORCE MEASURING SYSTEM

(71) Applicant: CONTITECH DEUTSCHLAND GMBH, Hannover (DE)

(72) Inventors: Max Konstantin WERHAHN, Hannover (DE); Peter Michael MARIENFELD, Hannover (DE); Moritz PLINKE, Hannover (DE)

(21) Appl. No.: 19/474,783

(22) PCT Filed: Mar. 27, 2024
(86) PCT No.: PCT/EP2024/058216

§ 371 Date: Oct. 13, 2025

(30) Foreign Application Priority Data
Apr. 13, 2023 (DE) 102023203349.6

(51) Int. Cl. A61B 5/11; A61B 5/00; A61B 90/00; G01L 1/20; G01L 1/26

(52) U.S. Cl. CPC A61B 5/112; A61B 5/6807; A61B 5/742; A61B 90/06; G01L 1/205; G01L 1/26; A61B 2090/064; A61B 2503/40; A61B 2560/0214; A61B 2562/063; A61B 2562/166; A61B 2562/227

(57) ABSTRACT

The present invention describes a reaction force measuring plate (1) for detecting the distribution of ground reaction forces across the ground contact area of the foot of a hoofed animal or the foot of a human being when it strikes the ground, with a preferably rigid carrier plate (3) with a first surface (3a) facing the ground during use and a second surface (3b) opposing the hoof or foot or vice versa, a plurality of flat force measurement sensors (5) fixed in position on the first surface (3a) of the carrier plate (3), and at least one temperature measuring sensor (11) which is designed and arranged to detect the temperature of at least one of the force measurement sensors (5), preferably several of the force sensors (5), and particularly preferably all of the force sensors (5).

The invention relates to a reaction force measuring plate which especially, is intended for use in a hoof boot, a hoof or a shoe and is suitable for determining a flat reaction force distribution when the hoof of a hoofed animal or the foot of a human strikes the ground. It also relates to a reaction force measuring system comprising such a measuring plate, and in addition, a horseshoe, hoof boot or shoe equipped with such a measuring plate is also claimed.

For the gait and health analysis of a horse or other hoofed animal (e.g., camel), it is desirable to measure the reaction forces generated when it strikes the substrate and/or ground, not only at a single point, but with a certain displacement across the entire contact area of the hoof. The measured values should enable conclusions to be drawn about the animal's state of health, e.g., with regard to lameness or strain on the musculoskeletal system.

While there are a whole range of solutions that are available for corresponding tasks in the fields of medical diagnostics, training condition analysis and rehabilitation in humans, the availability of corresponding systems for hoofed animals is limited. There is a commercially available product from Tekscan and other systems with a similar purpose, however these are based on measuring acceleration rather than reaction forces.

GB 2 482 192 B discloses the attachment of force sensors to a horseshoe for such purposes and the local storage of their signals and/or their transmission via a wireless transmitter to a remote receiving and evaluation station.

According to DE 10 2011 016 344A1 , force sensors are used in an elastomer body for a corresponding purpose. The elastomer body, which in turn is to be inserted into a hoof boot, is designed to additionally enable the use of commercially available resistive force sensors with their limited force measuring range for reaction force analysis in horses with their relatively high ground pressure.

From US 2020/319044 A1 teaches another system for the area-dissipated recording of reaction forces on the hooves of a hoofed animal, which transmits the sensor signals via a wireless transmitter to a remote evaluation unit. This system has a complex design consisting of a base plate and a ground plate, which are guided precisely in relation to each other and between which several force application cylinders and a plurality of resistive force measurement sensors are housed, as well as a microprocessor unit, a communication unit and a battery to supply force to the components. The function of the ‘force application cylinders’ is not apparent from the printed material, nor is the method of attachment to the hoof.

DE 10 2021 211 795.3 (unpublished) describes a reaction force measuring plate for recording the distribution of ground reaction forces across the ground contact area of the foot of a hoofed animal or the foot of a human being when it strikes the ground, with a rigid carrier plate with a first surface facing the ground during use and a second surface facing the hoof or foot, a plurality of flat force measurement sensors fixed in position on the first surface of the carrier plate, and several elastic force transmission studs, especially a plurality corresponding to the plurality of force measurement sensors, fixed to the free surfaces of the force measurement sensors.

One disadvantage of the known possibilities of this type for measuring the force of the foot of a hoofed animal or the foot of a human when it hits the ground, i.e., when it comes into contact with the ground, is that the measuring properties of the force measurement sensors used are temperature-dependent or can be influenced by the temperature in the immediate vicinity of the force sensor or its temperature. This can influence and/or falsify the detection of the measured value from which force values can be determined. This may affect the accuracy and/or quality of the measured value or the force value which is determined and/or detected from it. This can have a correspondingly deteriorating effect on the results of the evaluation of the force values.

One object of the present invention is to improve the possibilities for measuring the force exerted by the foot of a hoofed animal or the foot of a human being when it strikes the ground. In particular, temperature-related influences on force measurement should be avoided or at least reduced. This should be done in a way that is as simple, cost-effective, space-saving and/or weight-saving as possible. At the very least, an alternative to the familiar options should be created.

According to the invention, the object is solved by a reaction force measuring plate, a reaction force measuring system and a hoof boot, horseshoe or shoe with the features of the independent patent claims. Advantageous further embodiments are described in the subclaims.

As such, the present invention describes a reaction force measuring plate for recording the distribution of ground reaction forces across the ground contact area of the foot of a hoofed animal or the foot of a human being when it strikes the ground, with a preferably rigid carrier plate with a first surface facing the ground during use and a second surface opposing the hoof or foot or vice versa, a plurality of flat force measurement sensors fixed in position on the first surface of the carrier plate, and at least one temperature measuring sensor which is designed and arranged to detect the temperature of at least one of the force measurement sensors, preferably several of the force sensors, and particularly preferably all of the force sensors. The floor is a substrate that can basically be of any composition or made of any material.

The temperature measuring sensor is a temperature-sensitive element which has a temperature-dependent physical parameter, the value of which can be detected and converted into a corresponding temperature value. This allows the temperature of the temperature measuring sensor and/or its immediate surroundings to be detected and, a temperature value to be determined. Preferably, the temperature dependence is linear, at least in the relevant measuring range, which can make the detection of the sensor value and the determining of the temperature value in a way that is easier and/or more accurate. In any case, a PTC (Positive Temperature Coefficient) or an NTC (Negative Temperature Coefficient) element can be used as a temperature measuring sensor or as a temperature-sensitive element.

The present invention is based on the finding that the temperature of a force measurement sensor can influence its detected measured value and thus falsify the measurement result, as described above. This is usually more relevant, the more precise the measurement has to be.

According to the invention, the temperature of the force measurement sensors of the reaction force measuring plate according to the invention is therefore detected, so that the detected and/or determined temperature can be taken into account in the evaluation or determination of the force measuring values. This can also be done by detecting the temperature near and/or in the immediate vicinity of the force measurement sensors.

This can improve the accuracy of force measuring values. This can be done, for example, by means of a temperature-dependent factor, by means of which a sensor-detected physical quantity of the force measurement sensors is converted into a force value. Making the conversion factor not constant but temperature-dependent can allow the current temperature of the force measurement sensors to be taken into account when determining the force values.

The reaction force measuring plate according to the invention can have a carrier plate that is essentially both rigid and incompressible (but as the case may be, elastically flexible). Such a reaction force measuring plate has a simple design and is therefore inexpensive to manufacture, robust in practical use, and easy to handle. It also enables a sufficiently resolved recording of the reaction forces across the surface of the hoof with low leakage of the force and therefore high accuracy. It is also flat and lightweight in terms of structure, making it not only easy to use but also easy to transport.

Preferably, at least five force measurement sensors can be used. Preferably, at least, and particularly preferably exactly seven force measurement sensors can be used, which can be arranged along the edge. In any case, the force measurement sensors can be arranged at approximately equal distances from each other in the circumferential direction. This can enable representative recording of force values with limited cost.

Preferably, an additional force measurement sensor can be arranged centrally and/or at the edge of the heel of a person's foot and/or between the heel ends of the foot and/or hoof of a hoofed animal, which can increase and/or improve the significance of the detected measured values.

As explicitly stated and described above, the carrier plate can be used with the first surface facing the ground and the second opposite surface facing the hoof or foot. However, the arrangement can also be reversed, with the carrier plate facing the hoof or foot with the first surface in use and facing the ground with the second opposite surface. This can increase the utility and design possibilities.

In particular, the arrangement of the carrier plate with the first surface facing the hoof or foot during use is advantageous because the force measurement sensors and as the case may be, any elastic force transmission studs that may be available, then act against the hoof. Accordingly, the carrier plate does not need to be connected directly to the hoof or horseshoe. Instead, the fixation can be achieved, for example, by means of a hoof boot, which can also be formed by a protective element of the reaction force measuring plate itself, as will be described in more detail below.

According to one aspect of the invention, the temperature measuring sensor is arranged, preferably centrally, between the force measurement sensors. This allows a measured value to be detected that can represent the temperatures in the vicinity of the corresponding force sensors. This allows one temperature sensor to be used for several force measurement sensors, which can keep the cost, installation space and/or weight of the temperature sensor as low as possible.

According to a further aspect of the invention, the temperature measuring sensor is designed to extend over a surface, preferably as a long, preferably meander-shaped, electrical conductor. This can enable and/or promote a comparatively flat detection of an average temperature, which can be considered representative for several force measurement sensors.

According to a further aspect of the invention, the temperature measuring sensor is arranged on the first surface of the carrier plate. This can facilitate the application because the force sensors and the temperature measuring sensor can be arranged on the same side of the carrier plate.

According to a further aspect of the invention, the carrier plate has the shape of a closed horseshoe, circular ring or U or polygon, especially with a recess in the central area. This allows for adaptation for use on various hoofed animals or other vertebrates (including humans).

According to a further aspect of the invention, the force measurement sensors, together with associated sensor signal lines and optional power supply lines, are implemented on a continuous sensor carrier foil which is especially fixed to the first surface of the carrier plate. This can simplify implementation and production.

According to a further aspect of the invention, the temperature measuring sensor is further formed on the connected sensor carrier film, preferably as a printed electrical conductor, and preferably meandering. This can also be implemented accordingly for the temperature measuring sensor.

According to a further aspect of the invention, the force measurement sensors and/or the sensor carrier film are bonded to the carrier plate. This can be an easily implemented embodiment.

According to a further aspect of the invention, the force measurement sensors and/or the sensor carrier film are detachably fastened to the carrier plate, and especially inserted into suitable guides. If a high degree of configuration flexibility is required for certain applications, the force measurement sensors and/or the sensor carrier plate can also be alternatively attached in a way which allows them to be detached and, in particular, inserted into suitable guides or locked together. It goes without saying that such solutions are more complex to construct and potentially more susceptible to failure, so they are more likely to be suitable for special applications.

According to a further aspect of the invention, the effective area of the force measurement sensors is in the range between 0.5 cm² and 10 cm² , especially 2 cm² and 5 cm². It is understood that when using a relatively large number of force measurement sensors, and especially in configurations of the measuring plate intended for animals with relatively small hoof or foot areas, the effective area may be relatively small, whereas in configurations with a relatively small number of force measurement sensors and for animals with large detection areas, it may be closer to, or even above the preferred upper limit.

According to a further aspect of the invention, all force measurement sensors are substantially rectangular in shape and have the same geometric shape and effective area. This can facilitate the technologically easy and cost-effective manufacturing of the sensors and the configurability of different embodiments of the measuring plate.

According to a further aspect of the invention, the force measurement sensors are resistive-dielectric sensors, which, in particular, comprise a first conductive layer, a dielectric layer on top of this, which is surrounded and delimited by a spacer that determines the shape of the force measurement sensor, and a second conductive layer on the dielectric layer and the spacer. In addition to the structure mentioned here as a variant, such resistive force measurement sensors can also have a different structure, i.e., structures which are known as such. In principle, piezoelectric, capacitive, or inductive sensors or electroactive polymers can also be used in the reaction force measuring plate in addition to resistive dielectric sensors.

According to a further aspect of the invention, the carrier plate consists of organic sheet metal, spring steel or plastic. These may represent specific possibilities for implementation.

The present invention also relates to a reaction force measuring system with a reaction force measuring plate as described above and a wireless sensor signal transmission unit attached to it and connected to the force measurement sensors and the temperature measuring sensor in terms of signals, especially according to the Bluetooth standard, as well as a sensor signal receiver, evaluation and display device arranged remotely from the reaction force measuring plate, which comprises a wireless sensor signal receiving unit communicating with the reaction force measuring plate via the sensor signal transmission unit.

The present invention also relates to a hoof boot, horseshoe, or shoe to which a reaction force measuring plate as described above is fixed.

Ultimately, this proposes a horseshoe or hoof boot (for use on hoofed animals) or a shoe (for use on humans) that is equipped with a reaction force measuring plate on the underside. In the case of hoof boots, depending on their specific design, the measuring plate can be positioned on the inside and, as the case may be, fixed to the hoof or horseshoe. Although the invention is primarily intended for use with hoofed animals, it is also conceivable that the reaction force measuring plate according to the invention could be used for certain examinations on humans, for example to obtain information for rehabilitation or training purposes. This person will then wear shoes equipped with the measuring plate according to the invention.

An embodiment and further advantages of the invention are explained below in connection with the following figures. These show:

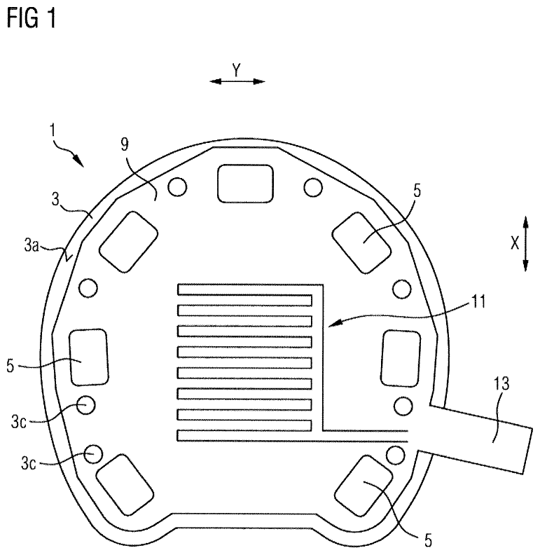

FIG. 1 the structure of an example reaction force measuring plate from a perspective view from below;

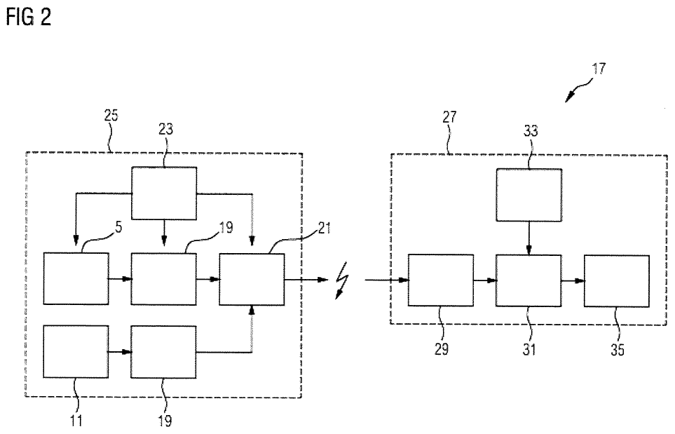

FIG. 2 a schematic diagram of an embodiment of the reaction force measuring system according to the invention, in the form of a block diagram; and

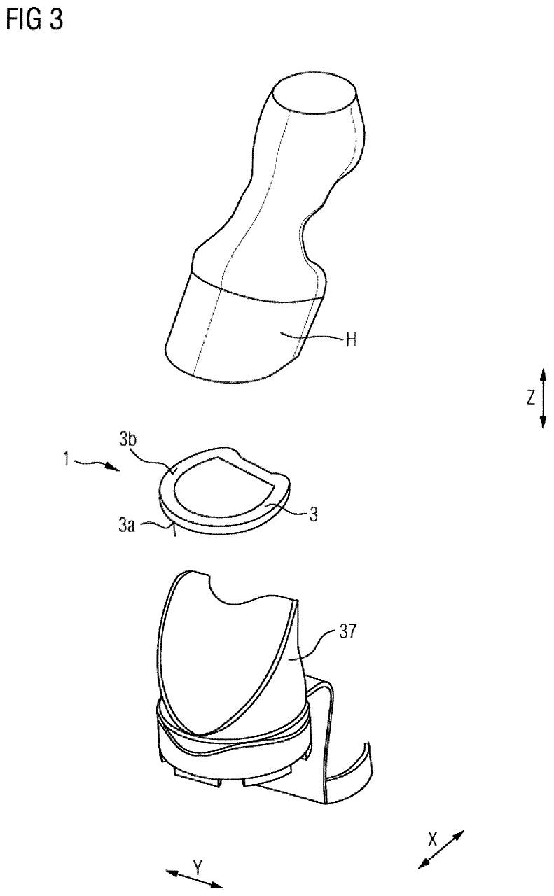

FIG. 3 a schematic representation of the attachment of the reaction force measuring plate according to the invention to a horse's hoof.

The above figures are described in Cartesian coordinates with a longitudinal direction X, a transverse direction Y perpendicular to the longitudinal direction X, and a vertical direction Z perpendicular to both the longitudinal direction X and the transverse direction Y. The longitudinal direction X can also be referred to as depth X, the transverse direction Y as width Y, and the vertical direction Z as height Z. The longitudinal direction X and the transverse direction Y together form the horizontal X, Y, which can also be referred to as the horizontal plane X, Y. The longitudinal direction X, the transverse direction Y, and the vertical direction Z can also be referred to collectively as spatial directions X, Y, Z or as Cartesian spatial directions X, Y, Z.

FIG. 1 shows, in a perspective view from below, the structure of an exemplary reaction force measuring plate 1 with a closed horseshoe-shaped rigid carrier plate 3, which has a first surface 3a and a second surface 3b. Seven resistive force measurement sensors 5 with a matching rectangular basic shape are attached to the first surface 3a at equal distances from each other. The free surface of each of the force measurement sensors 5 faces downward toward a ground surface (not shown) and thus away from, for example, the horse's hoof H. The force measurement sensors 5 can also be referred to as force sensors 5 or force sensor elements 5.

The force measurement sensors 5 are integrally implemented on a sensor carrier film 9, which also carries conductors (not shown) for connecting each sensor. The sensor carrier film 9, together with the force measurement sensors 5 formed thereon, can be manufactured using conventional means of printed circuit board technology, which are known to those skilled in the art. The sensor carrier film 9 is applied with its rear side to the first surface 3a of the carrier 3.

In the simplest case, the sensor carrier film 9 can be bonded to the carrier plate 3 using a suitable adhesive. In order to connect the reaction force measuring plate 1 to the hoof of the vertebrate or as the case may be, to an additional element located between them, the carrier plate in the design shown has holes 3c into which, for example, hoof nails can be driven or a screw can be inserted.

There is a temperature measuring sensor 11 exactly in the center between the force measurement sensors 5, which can also be referred to as a temperature sensor 11 or temperature sensor element 11. The temperature measuring sensor 11 is also implemented integrally on a sensor carrier film 9, specifically on the same side of the sensor carrier film 9 as the force measurement sensors 5. Both the force measurement sensors 5 and the temperature measuring sensor 11 can be contacted and read from outside by means of the conductor tracks and/or contacts (not shown) of a connector element 13 in the form of a plug outlet 13, as will be described in more detail below.

FIG. 2 is a schematic diagram of a reaction force measuring system 17, which can be formed, especially with a reaction force measuring plate according to FIG. 1.

The representation takes the form of a functional block diagram and is not intended to show the exact design of the system components.

In addition to the force measurement sensors 5 and temperature measuring sensor 11 with their connector outlet 13 already shown in FIG. 1, the reaction force measuring system 17 each comprises a sensor signal pre-processing unit 19 which is connected to the force measurement sensors 5 and the temperature measuring sensor 11 via the sensor signal lines and/or via the connector outlet 13 and serves to perform pre-processing and formatting of the sensor signals which is advantageous for external signal transmission.

On the output side, the sensor signal pre-processing units 19 are connected to a wireless sensor signal transmitter 21, which can operate according to the Bluetooth standard, for example, but also according to another standard for wireless message transmission, depending on the application. The above-mentioned components are assigned an energy source 23, which in the simplest case is a commercially available primary cell or a rechargeable battery. If a rechargeable battery is used, it can be assigned a charging socket (not shown in the figure) for recharging when installed.

All the above-mentioned components are advantageously arranged on the reaction force measuring plate 1, and especially protected in its interior, and are referred to here as hoof component 25. Specifically, the functional units of the hoof component 25 can all be realized on the sensor carrier film 9.

When the system is in use, the hoof component 25 is in wireless signal connection via the sensor signal transmitter 21 with a sensor signal receiver, evaluation and display device 27, which is placed away from the animal (or human) to be examined. The device 27 comprises a wireless sensor signal receiver 29 which is configured to communicate with the sensor signal transmitter 21 on the hoof component 25 and which feeds the received signals to a signal evaluation unit 31, where they are subjected to evaluation according to a program stored in a program memory 33. When evaluating the measured values of the force measurement sensors 5, the current temperature in the immediate vicinity of the force measurement sensors 5, which is detected by the temperature measuring sensor 11, can be taken into account in order to improve the accuracy of the determination of the forces.

Finally, a display unit 35 serves to display the evaluation results, for example for a therapist or trainer. The components of the sensor signal receiver, evaluation and display device can be implemented, for example, in a notebook, tablet or smartphone with a suitable evaluation app.

FIG. 3 shows an example of a possible use of the reaction force measuring plate 1 according to the invention, placed inside a hoof boot 37 of a commercially available type, which is attached to a hoof H of a horse. In the example shown, the reaction force measuring plate 1—depending on the design of the hoof boot—is temporarily fixed to the foot with a suitable, removable adhesive and/or bonding agent and then the hoof boot 37 is slipped over it, or the reaction force measuring plate 1 is inserted into the hoof boot and the hoof boot is then fixed to the hoof.

The implementation of the invention is not limited to the examples and aspects mentioned above, but is also possible in a variety of modifications that are within the scope of the appended claims.

### REFERENCE LIST (PART OF THE DESCRIPTION)

H hoof and/or horse hoof
X longitudinal direction; depth
Y transverse direction; width
Z vertical direction; height
X, Y horizontal; horizontal plane
1 reaction force measuring plate
3 carrier plate, sensor plate
3a first surface
3b second surface
3c openings
5 force measurement sensors; force sensors; force sensor elements
9 sensor carrier film
11 temperature measuring sensor; temperature sensor; temperature sensor element
13 connector element; plug outlet
17 reaction force measuring system
19 sensor signal pre-processing unit
21 wireless sensor signal transmitter
23 energy source
25 hoof component
27 sensor signal receiver, evaluation and display device
29 wireless sensor signal receiver
31 signal evaluation unit
33 program memory
35 display unit
37 hoof boot

1. A reaction force measuring plate for recording the distribution of ground reaction forces over the ground contact area of the foot of a hoofed animal or the foot of a human when it strikes the ground, with
a preferably rigid base plate with a first surface facing the ground during use and an opposite second surface facing the hoof or foot, or vice versa,
a plurality of flat force measurement sensors fixed in position on the first surface of the carrier plate,
at least one temperature measuring sensor which is designed and arranged to detect the temperature of at least one of the force measurement sensors, preferably several of the force sensors, and particularly preferably all of the force sensors.

2. The reaction force measuring plate according to claim 1,
wherein the temperature measuring sensor, is arranged, preferably centrally, between the force measurement sensors.

3. The reaction force measuring plate according to claim 1,
wherein the temperature measuring sensor extends over a surface, preferably designed as a long, preferably meander-shaped, electrical conductor.

4. The reaction force measuring plate according to claim 1,
wherein the temperature sensor is arranged on the first surface of the carrier plate.

5. The reaction force measuring plate according to claim 1,
wherein the carrier plate has the shape of a closed horseshoe, circular ring or U or polygon, especially with a recess in the central area.

6. The reaction force measuring plate according to claim 1,
wherein the force measurement sensors together with associated sensor signal lines and optional power supply lines are realized on a continuous sensor carrier film which is especially fixed on the first surface of the carrier plate.

7. The reaction force measuring plate according to claim 6,
wherein the temperature measuring sensor is further designed on the continuous sensor carrier film, preferably as a printed electrical line, and preferably meandering.

8. The reaction force measuring plate according to claim 1,
wherein the force measurement sensors and/or the sensor carrier film is/are bonded to the carrier plate.

9. The reaction force measuring plate according to claim 1,
wherein the force measurement sensors or the sensor carrier film are detachably fastened to the carrier plate, in particular inserted into suitable guides.

10. The reaction force measuring plate according to claim 1,
wherein the effective area of the force measurement sensors is in the range between 0.5 cm² and 10 cm², especially 2 cm² and 5 cm².

11. The reaction force measuring plate according to claim 1,
wherein all force measurement sensors are substantially rectangular in shape and have the same geometric shape and effective area.

12. The reaction force measuring plate according to claim 1,
wherein the force measurement sensors are resistive-dielectric sensors, which in particular comprise a first conductive layer, on this a dielectric layer which is surrounded and delimited by a spacer determining the shape of the force measurement sensor, and on the dielectric layer and the spacer a second conductive layer.

13. The reaction force measuring plate according claim 1,
wherein the carrier plate consists of organic sheet metal, spring steel or plastic.

14. A reaction force measuring system, with
a reaction force measuring plate according to claim 1 and
a wireless sensor signal transmitter attached thereto and connected to the force measurement sensors and the temperature measuring sensor, especially according to the Bluetooth standard, and
a sensor signal receiver, evaluation and display device arranged remotely from the reaction force measuring plate, which comprises a wireless sensor signal receiver communicating with the sensor signal transmitter on the reaction force measuring plate.

15. A hoof boot, horseshoe, or shoe to which a reaction force measuring plate according to claim 1 is fixed.
